(19) United States
(12) Patent Application Publication
(10) Pub. No.: US 2007/0190593 A1
(43) Pub. Date: Aug. 16, 2007

(54) Methods for detecting and characterizing microorganisms

(75) Inventors: Morteza Abbaszadegan, Chandler, AZ (US); Mohamad Elzein, Tempe, AZ (US); Absar Alum, Tempe, AZ (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Arizona Board of Regents, acting for and on behalf of Arizona State University

(21) Appl. No.: 11/524,176

(22) Filed: Sep. 20, 2006

Related U.S. Application Data
(60) Provisional application No. 60/719,047, filed on Sep. 20, 2005.

(51) Int. Cl. C12Q 1/04; G06F 19/00

(52) U.S. Cl. 435/34; 702/19

(57) ABSTRACT

The invention provides methods, reagents, and devices for rapid detection and characterization of live bacteria.

### CROSS-REFERENCE TO RELATED APPLICATIONS

This application claims priority to U.S. Provisional Patent Application No. 60/719,047 filed Sep. 20, 2005, incorporated by reference herein in its entirety.

### BACKGROUND OF THE INVENTION

Pathogenic and opportunistic bacteria can contaminate drinking water systems and other environments through activities including 1) noncompliance with standards, 2) microorganism regrowth in the water distribution networks, 3) catastrophic events including floods, toxic algal blooms, and contamination due to transient pressure drops, which leads to infiltration of groundwater into distribution network or incorrect cross-connections with sewer lines, and/or 4) intentional acts of bioterrorism. In the US, more than 80% of the population receives potable water delivered to their residences, their workplaces, and most of urban aquatic environments through networks of water distribution system pipelines. The engineered infrastructure of potable water distribution networks have been in place and operated for decades, with small incremental improvements in piping materials. Twenty four percent of U.S. waterbome disease outbreaks reported in community water systems in the 1990's were caused by contamination entering the water distribution system (i.e., not to poorly treated water at the centralized water treatment plant). Moreover, prolonged microbial growth can create biofilms, which can shelter against disinfection for some pathogenic microorganisms.

Most currently available sensors for continuous water quality monitoring focus on physicochemical parameters such as the measurement of chlorine residual, conductivity, pH value, temperature, and turbidity. These measures do not indicate if any specific hazards exist. Other detection methods for water quality monitoring have been developed utilizing the optical, electrochemical, biochemical and physical properties of microorganisms. However, each of these detection methods suffers drawbacks. For example, current photoacoustic spectroscopy (PAS) techniques are limited by its indirect detection scheme and lack of information on the types and status of microorganism in biofilms. Conventional culture methods take 24 to 48 hours for the detection and identification of any bacterial pathogen in water. Therefore, these methods have limited application in case of catastrophic events.

No currently marketed devices can produce a rapid, accurate, sensitive, real-time detection and characterization of microorganisms. Thus, there exists a need for sensors and sensor systems for the rapid and cost-effective detection and characterization of microorganism contamination in various environments.

### SUMMARY OF THE INVENTION

In a first aspect, the present invention provides methods for detecting bacteria in a sample comprising:
a. contacting a test sample with one or more fluorescence-emitting substrate selected from the group consisting of metabolic, proteolytic, glycosidic, and lipophylic substrates, under conditions suitable for the one or more substrates to be acted upon by bacterial enzymes present in the test sample that are specific for the one or more substrates;
b. detecting an activity over time of fluorescence emissions from one or more of the substrates that result from the contacting with the test sample; and
c. correlating the activities with the presence of live bacteria in the test sample.

In one embodiment of this first aspect, the methods further comprise:
d. contacting test samples identified as having live bacteria with one or more different fluorescence-emitting substrates selected from the group consisting of metabolic, proteolytic, glycosidic, and lipophylic substrates, under conditions suitable for the one or more different substrates to be acted upon by bacterial enzymes present in the test sample that are specific for the one or more different substrates;
e. detecting an activity over time of fluorescence emissions from one or more of the different substrates that result from the contacting with the test sample; and
f. correlating the activities with a bacterial type present in the test sample.

In another aspect, the invention provides methods for characterizing bacteria in a sample comprising:
a) contacting a test sample with one or more fluorescence-emitting substrates selected from the group consisting of metabolic, proteolytic, glycosidic, and lipophylic substrates, under conditions suitable for the one or more substrates to be acted upon by bacterial enzymes present in the test sample that are specific for the one or more substrates;
b) detecting an activity over time of fluorescence emissions from one or more of the substrates that result from the contacting with the test sample; and
c) correlating the activities with a bacterial type present in the test sample.

In a second aspect, the present invention comprises a reaction cell comprising two or more fluorescence-emitting substrates selected from the group consisting of metabolic, proteolytic, glycosidic, and lipophylic substrates. In a further embodiment, the reaction cell of the present invention further comprises a means for identification of the reaction cell.

In a third aspect, the present invention provides a detection device, comprising: a light source for illuminating a sample; an optical filter; a sample compartment comprising a reaction cell and a fluorescence intensifier; and a fluorescence collection device adapted for connection to a fluorescence detector, wherein the optical filter is interposed between the light source and the sample compartment, and wherein the fluorescence intensifier is interposed between the reaction cell and the fluorescence collection device.

In a fourth aspect, the invention provides a detection device, comprising: a light source for illuminating a sample; an optical filter; a sample compartment comprising a reaction cell and a means for intensifying fluorescence emitted from a sample in the reaction cell; a means for collecting fluorescence emitted from a sample in the sample compartment, wherein the optical filter is interposed between the light source and the sample compartment, wherein the means for intensifying fluorescence is interposed between the reaction cell and the means for collecting fluorescence, and wherein the means for intensifying the fluorescence emitted from the reaction cell is adapted for connection to a fluorescence detector.

Various embodiments of the invention will become evident from the following more detailed description of certain specific embodiments and the claims.

### BRIEF DESCRIPTION OF THE DRAWINGS

FIG. 1 provides an example of fluorescence response to bacterial number count for a pure culture of E. coli, where the fluorescence is generated using fluorescence-emitting proteolytic, glycosidic, and lipophylic substrates.

FIG. 2 provide microscopic images showing progression in E. coli biofilm formation on flat surfaces over a period of six weeks.

FIG. 3 provides an example of fluorescence response (FI, or fluorescence intensity) to bacterial number count for a pure culture of E. coli, where the fluorescence is generated using fluorescence-emitting proteolytic, glycosidic, and lipophylic substrates.

FIG. 4 provides an example of fluorescence response (FI, or fluorescence intensity) to bacterial number count for a pure culture of Mycobacterium phlei, where the fluorescence is generated using fluorescence-emitting proteolytic, glycosidic, and lipophylic substrates.

FIG. 5 provides an example of fluorescence response (as indicated by relative fluorescence) to over time for a pure culture of Novosphingobium, where the fluorescence is generated using fluorescence-emitting metabolic, proteolytic and glycosidic substrates.

### DETAILED DESCRIPTION OF PREFERRED EMBODIMENTS

The methods of the present invention take advantage of the variations in the biochemical reaction processes between living and dead bacteria as well as between various genera of bacteria to detect and/or characterize bacteria in test samples.

In a first aspect, the present invention provides methods for detecting bacteria in a sample comprising:
a. contacting a test sample with one or more fluorescence-emitting substrate selected from the group consisting of metabolic, proteolytic, glycosidic, and lipophylic substrates, under conditions suitable for the one or more substrates to be acted upon by bacterial enzymes present in the test sample that are specific for the one or more substrates;
b. detecting an activity over time of fluorescence emissions from one or more of the substrates that result from the contacting with the test sample; and
c. correlating the activities with the presence of live bacteria in the test sample.

Any type of bacteria for which suitable substrates are available can be detected or characterized (see below) using the methods of the invention.

As used herein, a “test sample” is any sample in which live bacteria may be present and from which fluorescence can be detected. Non-limiting examples of test samples are liquids, biofilms and gas samples. Liquid samples include, but are not limited to, water samples (including but not limited to water samples from ponds, streams, lakes, oceans, seas, wastewater, reservoirs, drinking water, water distribution pipeline, etc.) as well as any other types of liquid, such as body fluid samples (including but not limited to blood, urine, sweat, vaginal secretions, sputum), beverage samples, liquid medicine samples, as well as any other liquid borne samples. A test sample does not need to initially be a liquid, but instead can be derived from a non-liquid sample (ie: solid or gas) that is subsequently placed in a liquid prior to detection. In one non-limiting example, a food sample can be swabbed and the swab can subsequently be inserted into a liquid (ie: water, buffer, etc.) to generate a liquid test sample from a solid. Those of skill in the art will understand how to prepare other such non-liquid samples for use as a test sample based on the teachings herein. The non-liquid test samples can be any sample of interest, including but not limited to, food samples, environmental samples (from, e.g., medical centers such as linens, medical devices, etc.); pharmaceutical facilities (from, e.g., manufacturing or processing lines); food production facilities; livestock facilities; solid waste samples and diagnostic samples.

The test sample can also comprise bacterial biofilm. In one non-limiting example, biofilm is collected and placed in a liquid, such as water or buffer solution. Examples of gaseous test samples include, but are not limited to, air, air filters, air duct and breath samples

The test sample can be used as obtained, or can be processed in any way suitable for use with the methods of the invention. In one embodiment, the test sample is extracted from a larger volume, improving detection limits in the resulting test sample relative to the original larger volume. In one non-limiting example, a test sample is concentrated by collecting a liquid sample and concentrating it on a filter by passing the liquid sample through the filter. Other methods of concentrating the test sample known to those of skill in the art, such as centrifugation, can also be used to extract the test sample from a larger volume. The test sample may optionally be processed by placing it in a vacuum to reduce adsorbed gas, thereby improving detection limits.

In a further embodiment, the test sample is substantially translucent. As will be understood by those of skill in the art based on the discussion above, such substantially translucent test samples can be prepared from virtually any starting sample by standard techniques, including but not limited to, concentration or extraction of test samples from non-translucent materials using filters, centrifugation or any other suitable method followed by suspension or dissolution in a translucent liquid.

As used herein, a “substrate” is a compound on which a relevant enzyme can act, wherein the enzymatic activity (i.e., the enzyme/substrate reaction) results in a change in fluorescence emitted from the substrate as compared to the fluorescence emitted from the substrate in the absence of the enzyme. Since such a change in fluorescence only occurs in the presence of active enzyme, the substrates are specific for live bacteria. The substrates can be provided in any suitable format, including but not limited to, powders, liquids (as solutions or suspensions), capsules, liquid granules, coatings (such as coatings on a reaction cell), or any combination thereof.

As used herein, “proteolytic” substrates are those substrates that are acted upon by one or more bacterial protease. A “protease” is an enzyme that cleaves peptide bonds between amino acids. Non-limiting examples of bacterial proteases include aminopeptidases and carboxylases. A non-limiting example of a suitable proteolytic substrate includes, but is not limited to, L-Leucine-β-Naphthylamide (LLβN) or alanine or any other amino acids that can be tagged with any fluorescing molecules. Bacterial L-Leucine aminopeptidase hydrolyzes LLβN and releases the β-Naphthylamine (βN) fluorescent molecule through the following reaction:
LLβN+Bacterial L-Leucine Aminopeptidase→L-Leucine+Bn

As used herein, “glycosidic” substrates comprise glycosidic bonds and are acted upon by one or more bacterial enzymes that act on glycosidic bonds. Non-limiting examples of such bacterial glycosidic enzymes include glucosidases (β-D-glucosidase) and galactosidases (e.g., β-D-galactosidase). Non-limiting examples of suitable substrates for a glycosidic enzyme include, but are not limited to, o-nitrophenyl β-D-galactoside, 4-Methylumbelliferyl β-D-glucuronide hydrate (for β-D-glucuronidase activity). and 4-Methylumbelliferyl β-D-glucopyranoside (MUF). Bacterial β-D-glucosidase hydrolyzes MUFβ and releases the 4-Methylumbelliferone (MUF) fluorescent molecule through the following reaction:
MUF-62 +Bacterial β-D-Glucosidase→β-D-Glucoside+MUF

As used herein, “lipophylic” substrates are those substrates that are acted on by one or more bacterial enzymes that act on lipids, including but not limited to bacterial lipases. Non-limiting examples of suitable substrate for a bacterial lipophylic enzyme include, but are not limited to, N-phenyl-2-naphthylamine (NPN), 4-methylumbelliferyl-heptanoate, 4-Methylumbelliferyl butyrate, 4-Methylumbelliferyl oleate, 4-Methylumbelliferyl palmitate, 4-Trifluoromethylumbelliferyl oleate, Fluorescein dibutyrate, Fluorescein dilaurate and Indoxyl acetate.

As used herein, “metabolic” substrates are those that are acted upon by bacterial enzymes involved in biochemical modification of chemical compounds in living organisms, including but not limited to carbohydrate metabolism, fatty acid metabolism, protein metabolism and nucleic acid metabolism. Non-limiting examples of metabolic enzymes include phosphatases, dehydrogenases and esterases. Examples of suitable substrate for various metabolic enzymes include, but are not limited to, 4-Methylumbelliferyl Phosphate (4-MUP) (alkaline phosphatases), fluorescein diacetate (esterases) and 4-methylumbelliferyl acetate (esterase). In a specific embodiment, fluorescein diacetate or 4-methylumbelliferyl acetate is used as the substrate in the detection step.

In one embodiment, the substrates used to practice the methods of the invention are those substrates that can be those acted on by enzymes expressed by many, if not all of bacterial strains for which detection is desired. Such substrates are acted on by the bacterial enzymes at different levels in different bacteria, thus leading to different rates of change of the fluorescence emissions. These different rates of change of the fluorescence emissions in response to the different substrates allows for characterization of the bacteria.

The substrates can emit fluorescence by any mechanism, including autofluorescence or emission in response to absorbance of light of an appropriate wavelength. Thus, the substrates can comprise, for example, fluorescently labeled substrates and autofluorescent substrates. Non-limiting examples of fluorescently labeled substrates include substrates labeled with dyes, quantum dots and fluorescently labeled molecules (including but not limited to fluorescently labeled antibodies and ligands). Examples of fluorophores and dyes that can be used in the present invention include, but are not limited to, fluorescein, Texas Red, derivates of coumarin, rhodamine, naphthylamine and methylumbellyferone. Non-limiting examples of autofluorescent substrates include naturally autofluorescent substrates and those expressed as hybrid molecules with autofluorescent proteins, including but not limited to green fluorescent protein and related fluorescent proteins.

In the present invention, the “contacting” of substrate and test sample occurs under suitable conditions that allow the enzymes in the test sample to act on the one or more substrate. Such conditions can be determined by those of skill in the art based on the teachings herein. In one embodiment, the contacting occurs at a controlled temperature to, for example, optimize the enzyme-substrate reaction, optimize the fluorescence intensity, speed up or slow down the enzyme-substrate reaction, or for any other effect that may be desired in detecting the increased activity over time of fluorescence emissions. In various embodiments, the methods are conducted at temperatures between 5° C. and 35° C. In another embodiment, the contacting occurs in a controlled light environment so as to minimize the effect of exogenous light on the fluorescence emissions. In yet another embodiment of the methods of the present invention, the contacting step further comprises the addition of one or more compounds or performance of one or more processes to improve light transmission. Such compounds to improve light transmission are known to those of skill in the art and include, without limitation, compounds that decrease turbidity. Such processes to improve light transmission are known to those of skill in the art and include, without limitation, sedimentation or some sample processing techniques such centrifugation or filtration can be applied to decrease turbidity.

The test sample and one or more substrates can be mixed prior to the detection step or during the detection step. Such mixing includes, but is not limited to combining, blending, stirring, etc., and can be accomplished by any suitable means known to those of skill in the art, including but not limited to, stir bars, air flow, or other means of mechanical mixing or agitation.

It will be understood by those of skill in the art that other components may be combined with the test sample and one or more substrates prior to detection, including but not limited to buffers and diluents.

Contacting of the test sample and the one or more substrates can occur in a manner suitable for use. In one embodiment, contacting occurs in a reaction cell. In another embodiment, contacting occurs prior to placement of the resulting combination in a reaction cell. In one non-limiting example, the test sample is concentrated on a filter, and the filter is inserted in the reaction cell (e.g., cuvette) for detecting an increased activity over time of fluorescence emissions from one or more of the substrates. The filter may be a soluble filter, which dissolves in the reaction cell after insertion in the reaction cell, thereby liberating the test sample from the filter.

In embodiments where the contacting step in the method for detecting bacteria in a sample of the present invention occurs in a reaction cell, the one or more substrates can be provided in any suitable format, including but not limited to, coatings on the reaction cell.

In one embodiment, the contacting of substrate and test sample occurs once and the fluorescence emission is monitored for a suitable amount of time, for example 1 minute to 120 minutes, at any desired interval. In various embodiments, the fluorescence emission is monitored from 1 minute-120 minutes, from 5 minutes-120 minutes, from 5 minutes-90 minutes, from 10 minutes-120 minutes, from 10 minute-90 minutes, from 20 minutes-120 minutes, or from 20 minutes to 90 minutes.

The methods for detecting bacteria in a sample of the present invention can comprise analysis of a single test sample. Alternatively, the methods for detecting bacteria in a sample of the present invention can comprise detection of multiple test samples, either simultaneously (in the same reaction cell or in parallel) or sequentially (in the same reaction cell or in parallel).

When the fluorescence emission is monitored at intervals over a suitable amount of time, the contacted substrate and test sample can remain in the detection equipment, or the contacted substrate and test sample can be moved into and out of the detecting equipment at the desired intervals for evaluation of the fluorescence intensity. When the evaluation of the fluorescence intensity for a given test sample and substrate is detected by moving the sample into and out of the detection equipment, the detection equipment can be used to evaluate the fluorescence intensity for one or more samples by staggering the detection intervals such that only a single sample needs to be in the detection equipment at any one time.

As used herein, “activity over time of fluorescence emissions” encompasses any basis for detecting fluorescence emissions over a period of time. The activity can be an increase or decrease in fluorescence intensity, or no change may be observed. In one embodiment, the activity over time of fluorescence emissions is measured by the slope over time of fluorescence emissions from the substrates that result from the contacting with the test sample. In a further embodiment, the slope of fluorescence emissions that is used to detect the activity over time of fluorescence emissions is the linear slope of fluorescence emissions. As used herein, “linear slope” means the linear portion of a curve derived from measuring fluorescence emission over time. As the fluorescence emission in the methods of the invention are dependent on the enzymatic activity of live bacteria, using the linear slope of the fluorescence emission over time minimizes the role of bacterial replication in increased fluorescence emissions over time. In another embodiment, the activity over time of fluorescence emissions is measured by calculating the area under the curve for fluorescence emission over any given time interval. In a further embodiment, the activity over time of fluorescence emissions is measured by integrating the emitted fluorescence signals over a known period of time. The activity over time of fluorescence emissions can be measured any detection algorithms known to those of skill in the art.

As used herein, “detecting” the activity over time of fluorescence emissions is accomplished using any suitable means for detection of fluorescence emissions from the substrates according to one or more of the various embodiments disclosed herein, including but not limited to a spectrophotometer.

As used herein, “correlating the activities” involves comparing fluorescence emission activity to one or more standards so as to determine whether the activity detected in the test sample is due to the presence of live bacteria. Standards include, but are not limited to, internal controls and previously identified fluorescence emission activity for various bacteria. In one embodiment, such standards are those stored in a database that have been generated using the same substrates and may have been generated using the same detection device.

The foregoing method of present invention can be used to detect and differentiate live bacteria from dead bacteria because the substrates are acted upon only in the presence of live bacteria with active relevant enzymatic activity. In addition this method of the present invention can be used to identify a profile or “fingerprint” for bacteria based on the readings obtained (the activity over time of fluorescence emissions from one or more of the substrates) for each of the one or more substrates.

When autofluorescent substrates are used, detection does not require use of an excitation wavelength to stimulate fluorescence. When fluorescently labeled, non-autofluorescent substrates are used, the detecting comprises exposing the combined test sample and substrate to light of an appropriate wavelength that encompasses the excitation wavelength required to generate fluorescence emission by the specific substrate or substrates used in the method. For example, when 4-methylumbelliferyl β-D-glucopyranoside is used to detect glycosidic activity, the excitation wavelength preferably includes light of about 360 nm wavelength. In various embodiments disclosed below, the methods may utilize multiple excitation wavelengths, and detecting may comprise detecting increased activity of fluorescence emissions over time at multiple wavelengths, corresponding to the emission spectra of the substrates used in a particular method.

In various embodiments of the method for detecting bacteria in a sample of the present invention, the test sample is contacted with two, three, or more of the fluorescence-emitting substrates. In these embodiments, the fluorescence-emitting substrates are selected from the group consisting of metabolic, proteolytic, glycosidic, lipophylic substrates and any combinations thereof. By way of a non-limiting example, two different metabolic substrates can be used, or two different metabolic substrates and one lipophylic substrate can be used. Many further such embodiments will be apparent to those of skill in the art. In these embodiments, the test sample can be contacted simultaneously (in the same reaction cell or in parallel) or sequentially (in the same reaction cell or in parallel) with the two or more fluorescence-emitting substrates, or any combination thereof. As used herein, “sequentially” means that at least two separate detection events occur in the practice of the method. By way of non-limiting example, if three fluorescence-emitting substrates are used in the practice of the invention, the detection step can comprise contacting substrates 1 and 2 simultaneously with the test sample while contacting substrate 3 with the test sample subsequently. Many further such embodiments will be apparent to those of skill in the art.

In embodiments where two or more substrates are contacted simultaneously with the test sample, the fluorescence emissions from the different substrates can be optically distinguishable, or can be optically indistinguishable. By using fluorescence-emitting substrates that emit fluorescence at spectrally distinguishable wavelengths, the activity over time of fluorescence emissions from the two or more fluorescence-emitting substrates can be detected independently. Alternatively, by using fluorescence emitting substrates that emit fluorescence at spectrally indistinguishable wavelengths, the activity over time of fluorescence emissions from the two or more fluorescence-emitting substrates can be detected as a single measurement, thus minimizing variations due to the use of different fluorescence-emitting molecules and multiple excitation-emission wavelengths. Those of skill in the art will understand that where three or more substrates are used, any combination of spectrally distinguishable/indistinguishable substrates can be used. Any of these techniques can be used to provide detection and/or characterization of live bacteria present in the test sample.

Contacting occurs in a reaction cell, which is any container suitable for use with the methods of the present invention. Non-limiting examples of reaction cells include cuvettes, capillary tubes and flow cells. In one embodiment, the reaction cell is disposable. Such disposable reaction cells can be made of any suitable material, including but not limited to, plastic. In another embodiment, the reaction cell is reusable. Such reusable reaction cells may be made of any material suitable for multiple or long-term use, including but not limited to, glass and quartz. In a further embodiment, reaction cells are sterilizable to prevent cross contamination of test samples, which could lead to erroneous results including, but not limited to, false-positive results when detecting bacterial contamination, over estimation of bacterial contamination and mis-characterization of bacterial contaminants in a particular test sample. Reaction cells can be sterilized using any means known to one of skill in the art, including but not limited to ozone treatment, UV treatment, heat treatment or other chemical treatment capable of sterilizing the reaction cell.

In a further embodiment, the methods of the invention can be used for characterizing bacteria in a sample. In these embodiments, the methods of the first aspect further comprise:
d. contacting test samples identified as having live bacteria with one or more different fluorescence-emitting substrates selected from the group consisting of metabolic, proteolytic, glycosidic, and lipophylic substrates, under conditions suitable for the one or more different substrates to be acted upon by bacterial enzymes present in the test sample that are specific for the one or more different substrates;
e. detecting an activity over time of fluorescence emissions from one or more of the different substrates that result from the contacting with the test sample; and
f. correlating the activities with a bacterial type present in the test sample.

As used herein, the “one or more different fluorescence-emitting substrates” refer to at least one fluorescence-emitting substrate used in steps d and e of the methods of the present invention that is not the same as the one or more fluorescence-emitting substrates used in steps a and b of the detection methods disclosed above. By way of a non-limiting example, if only one fluorescence-emitting substrate is used for bacterial detection in steps a-c of the detection method disclosed above, then the same fluorescence-emitting substrate could not be used as the only fluorescence-emitting substrate to characterize the bacteria in steps d-f of the bacterial characterization method. Alternatively, if only one fluorescence-emitting substrates is used as the one or more fluorescence-emitting substrate to detect bacteria in steps a-c of the detection method disclosed above, then the same fluorescence-emitting substrate could be used as a fluorescence-emitting substrate to characterize the bacteria in steps d-f of the methods of the present invention so long as at least one other fluorescence-emitting substrate is used in steps d-f of the bacterial characterization method.

In another aspect, the invention provides methods for characterizing bacteria in a sample comprising:
a) contacting a test sample with one or more fluorescence-emitting substrates selected from the group consisting of metabolic, proteolytic, glycosidic, and lipophylic substrates, under conditions suitable for the one or more substrates to be acted upon by bacterial enzymes present in the test sample that are specific for the one or more substrates;
b) detecting an activity over time of fluorescence emissions from one or more of the substrates that result from the contacting with the test sample; and
c) correlating the activities with a bacterial type present in the test sample.

In various embodiments of these bacterial characterization methods, 2, 3, 4, 5, or more different fluorescence-emitting substrates selected from the group consisting of metabolic, proteolytic, glycosidic, and lipophylic substrates are contacted with the test sample. All embodiments of the detection methods disclosed apply to these characterization methods as well.

The characterization methods can be used, for example, to identify a bacterial type present in the test sample, and to identify a profile or “fingerprint” for bacteria based on the readings obtained (the activity over time of fluorescence emissions from one or more of the substrates) for each of the one or more substrates used to detect live bacteria and for each of the one or more different substrates. Such profiles can then be used, for example, to characterize bacterial types present in a test sample and also for populating a database for profiles to be used as controls in future assays.

The methods of the invention and all embodiments thereof can further comprise additional steps as desired. For example, the test sample can also be contacted with one or more other compounds that serve to assist in identification and/or characterization of bacteria present in the test sample. Such compounds can include, but are not limited to, antibodies to bacterial proteins, ligands that bind to bacterial receptors, and reporters for proteins specific to a given bacterial strain or species. Thus, such further compounds can themselves be labeled, so long as distinguishable from the fluorescence emitted from the substrates used. Alternatively, the one or more further compounds can be detected using any other means available, such as calorimetric means.

In a preferred embodiment of each of the various embodiments of the methods disclosed above, the method is automated. As used herein, “automated” means computer controlled. In one embodiment, the various fluorescence emission detection and correlation steps are automated, and the resulting information obtained from the methods is automatically used to populate a database. In further embodiments, other steps in the method can also be automated.

In a third aspect, the invention comprises reaction cells comprising one or more fluorescence-emitting substrate selected from the group consisting of metabolic, proteolytic, glycosidic, and lipophylic substrates, wherein the one or more substrates can be used in the reaction cell to carry out the methods of the present invention. In a preferred embodiment, the reaction cell comprises two, three, four, or more fluorescence-emitting substrates. The reaction cell can be either a disposable reaction cell or a reusable reaction cell. The fluorescence-emitting substrates can provided in any suitable format, such as powders, liquids (as solutions or suspensions), capsules, liquid granules, coatings on the reaction cell, or any combination thereof. When the reaction cell is a reusable reaction cell, the fluorescence-emitting substrates are preferably provided as powders, liquids (as solutions or suspensions), capsules, liquid granules or any combination thereof such that the two or more fluorescence-emitting substrates can be replenished when the reaction cell is reused. In a further embodiment, the reaction cell comprises one or more means for identification of the reaction cell, including but not limited to mechanical means, electronic means including but not limited to a Radio Frequency Identification (RFID) tag or a barcode, or chemical means including but not limited to dyes. The electronic means of identification can comprise a unique identifier as well as relevant information about the reaction cell and its contents before and after the test. The chemical means of identification would provide a simple and rapid means to provide an indication of authenticity of the reaction cell. The means of identification would prevent the use of non-authentic reaction cells, such as reaction cells without the fluorescence-emitting substrates.

In a fourth aspect, the invention provides a detection device comprising a light source for illuminating a sample; an optical filter; a sample compartment comprising a reaction cell and a fluorescence intensifier; and a fluorescence collection device adapted for connection to a fluorescence detector, wherein the optical filter is interposed between the light source and the sample compartment, and wherein the fluorescence intensifier is interposed between the reaction cell and the fluorescence collection device. The light source can be any light source suitable for use fluorescence detection, including but not limited to xenon light sources and light emitting diodes (LEDs).

The optical filter allows for the selection of an excitation wavelength as desired, and can be any optical filter including but not limited to low-pass or band-pass filter. In one embodiment, the optical filter is an adjustable band-pass linear variable filter, 300-750 nm. The optical filter is operably coupled to the light source. In one embodiment, the light source is coupled to the optical filter via a light adaptor. In one embodiment, the light adaptor comprises an optical fiber and any suitable connectors for coupling to the light source and the optical filter. Suitable connectors include, for example, SMA 905 connectors, Premium SMA 905 connectors, Laser SMA Connectors, ST Connectors and FC Connectors. The optical filters are arranged to direct filtered light on the sample compartment. In one embodiment, a collimating lens is interposed between the optical filter and the sample compartment. The collimating lens focuses the light path and increases the light throughput. The collimating lens can be connected to the optical filter and the sample compartment using any suitable connector, including those mentioned above for connecting the light source to the optical filter. Any suitable collimating lens can be used in the present invention.

The sample compartment comprises a reaction cell, such as those disclosed above in the methods and reaction cells of the invention. The sample compartment is of any suitable configuration and material to contain the reaction cell. In one embodiment, the reaction cell is a cuvette and the sample compartment is a cuvette holder. The sample compartment may also comprise a means for enhancing the signal emitted from a sample in the reaction cell by collecting the fluorescence that would otherwise be lost, by reflecting the excitation energy back through the sample or by both of these mechanisms. This means for enhancing the signal emitted from a sample in the reaction cell is interposed between the reaction cell and the means for collecting fluorescence. In one embodiment, the sample compartment comprises a fluorescence intensifier. In another embodiment, the fluorescence intensifier and the means for intensifying fluorescence emitted from a sample in the reaction cell is a mirror attached to at least one of the walls of the reaction cell. In another preferred embodiment, the fluorescence intensifier and the means for intensifying fluorescence emitted from a sample in the reaction cell is a sample compartment with one or more reflection mirrors attached to at least one of the walls of the sample compartment. In another embodiment, the fluorescence intensifier and the means for intensifying fluorescence emitted from a sample in the reaction cell is a cuvette holder with one or more reflection mirrors attached to at least one of the walls of the cuvette holder. In this embodiment, the reflection mirrors can be mirrored screw plugs, and the reflection mirrors can be, for example, UV-enhanced aluminum-coated mirrors or gold coated mirrors.

The fluorescence emitted from the sample is collected for transmission to a fluorescence detector. In one embodiment, a means for collecting fluorescence emitted from a sample in the sample compartment, adapted for connection to a fluorescence detector is provided. The means for collecting fluorescence emitted from a sample in the sample compartment can be any fluorescence collection device adapted for connection to a fluorescence detector, including but not limited to an optical fiber. The optical fiber can be connected using any suitable connector, including those mentioned above for connecting the light source to the optical filter.

The detection device optionally comprises a fluorescence detector connected to the fluorescence collection device or the means for collecting fluorescence emitted from a sample in the sample compartment. In one embodiment, the fluorescence detector is a high resolution spectrometer with high enough resolution to detect and differentiate weak fluorescence signals.

In a further embodiment, the fluorescence detector, such as a spectrometer, is operably linked to a processing device comprising a processor with database storage capability. The processing device allows the output signals of the detection device to be processed and stored in a database with simple digital circuitry and software.

In another embodiment, the detectable device of the present invention comprises a means for identification of the reaction cell within the sample compartment. As discussed above, the reaction cells of the present invention can comprise any means for identification. Thus, when the reaction cell comprises a means for identification, the detection device of the present invention preferably comprises a means for identifying the reaction cell in the device, including but not limited to an RFID reader, a barcode reader, or a colorimetric detector.

In yet another embodiment, the detection device is automated, such that the device can automatically perform test sample extraction and preparation tasks, as well as substrate introduction tasks. Other tasks can also be automated, including moving a test sample into and out of the sample holder for periodic evaluation of fluorescence emissions, moving multiple test sample into and out of the sample holder for sequential evaluation of fluorescence emissions, discarding the processed sample, and sterilizing the reaction cells after a test sample is discarded.

In a further aspect, the present invention provides computer readable storage media, for automatically carrying out the methods of the invention on a detection device, such as a fluorescence detection device. As used herein the term “computer readable medium” includes magnetic disks, optical disks, organic memory, and any other volatile (e.g., Random Access Memory (“RAM”)) or non-volatile (e.g., Read-Only Memory (“ROM”)) mass storage system readable by the CPU. The computer readable medium includes cooperating or interconnected computer readable medium, which exist exclusively on the processing system or be distributed among multiple interconnected processing systems that may be local or remote to the processing system.

As used herein, “fluorescence detection device” means a device capable of carrying out the fluorescence detection required to carry out the methods of the present invention, including, but not limited to, the detection devices disclosed herein.

In a further aspect, the present invention provides fluorescence detection devices (defined as above) that comprise computer readable storage media (also as described above) for carrying out the methods of the invention.

This invention is further illustrated by the following experimental investigations and examples, which should not be construed as limiting the present invention to these precise embodiments. The contents of all references, patents and published applications cited throughout this application are hereby incorporated by reference herein. As used herein, the singular forms “a”, “an” and “the” include plural referents unless the context clearly dictates otherwise.

### EXAMPLE 1

Fluorometric Assay Reagents

Reagents for Proteolytic Assays

For the proteolytic enzymatic assays, β-Naphthylamine standard (βN) (Sigma-N8381) and L-Leucine β-Naphthylamide Aminopeptidase substrate (LLβN) (Sigma-L1635) were dissolved in analytical grade Ethanol. All working standard and reagent solutions were adjusted to pH 7.5 using HEPES buffer. The βN working standard concentrations were prepared from the stock 40 mM βN solution in freshly prepared HEPES buffer (pH 7.5) and analyzed immediately.

Standard β-Naphthylamine (βN) Preparation

An amount of 0.090 g βN was weighed, transferred to a sterile 15 mL centrifuge tube, and dissolved in 15.71 mL pure Ethanol. The content was mixed at room temperature until dissolved. The tube was capped and labeled as 40 mM βN Stock standard solution. The solution was protected from light and stored at 4° C.

Substrate L-Leucine β-Naphthylamide Aminopeptidase (LLβN) Preparation

An amount of 0. 100 g LLβN was weighed and dissolved in 9.75 mL Ethanol in a sterile 15 mL centrifuge tube. The content was mixed at room temperature until dissolved. The tube was capped and labeled as 40 mM LLβN Stock substrate solution. The solution was protected from light and stored at 4° C.

Substrate L-Leucine-4-Methylumbelliferone Preparation

An amount of 5.0 mg L-Leucine-4-Methylumbelliferone was weighed and dissolved in 5.0 mL Ethanol in a sterile 15 mL centrifuge tube. The content was mixed at room temperature until dissolved. The tube was capped and labeled as 3.9 mM L-Leucine-4-Methylumbelliferone Stock substrate solution. The solution was protected from light and stored at 4° C.

Reagents for Glycosidic Assays

For the glucopyranosidase enzymatic activity, 4-Methylumbelliferone standard (MUF) (Sigma-M 1381) and 4-Methylumbelliferyl β-D-glucopyranoside substrate (MUFβ) (Sigrna-69591) were first dissolved in thyleneglycolmonomethylether (2-Methoxyethanol) (Sigma-284467), and then diluted with nanopure grade sterilized water (1:1, v:v). The crystals had to be completely dissolved before adding water. All working standards and reagents were adjusted to pH 7.5-10, preferably to pH 8 using freshly prepared glycine buffer solutions. The standard MUF concentrations were prepared directly before use.

Standard 4-Methylumbelliferone (MUF) Preparation

An amount of 0.141 mg MUF standard was weighed and transferred to a sterile 15 mL centrifuge tube. This was completely dissolved first in 10.0 mL of pure 2-Methoxyethanol. The tube was capped and labeled as 0.08 mM MUF. The solution was protected from light and stored at 4° C.

Substrate 4-Methylumbelliferyl β-glucopyranoside (MUF-β) Preparation

A weighed amount of 0.030 g MUF-β substrate was transferred to a sterile 15 mL centrifuge tube and completely dissolved in 5.0 mL pure 2-Methoxyethanol. A volume of 5.0 mL nanopure grade sterilized water was added. The solution was mixed, capped and labeled 8.87 mM MUF-β stock solution. The solution was protected from light and stored at 4° C.

Reagents for Lipophylic Assays

For the lipophylic activity, N-Phenyl-2-Naphthylamine (NPN) hydrophobic binding agent/probe (Sigma-178055) was prepared in analytical grade ethanol. Working solutions were adjusted to pH 7.5 using HEPES buffer. The NPN stock solution was prepared by dissolving 0.110 g NPN to 10.0 mL of analytical grade Ethanol in a sterile 15 mL centrifuge tube. The tube was capped, labeled as 50 mM NPN Stock solution. The solution was protected from light and stored at 4° C.

Reagents for Metabolic Assays

Substrate Fluoresceine Diacetate (FDA) Preparation

The substrate Fluorescein Diacetate (FDA), (Di-O-Acetylfluorescein or 3,6-Diacetoxyfluoran) (Si gma-31545), was used to study the bacterial metabolic activities through their intracellular interactions and membrane permeability. The stock substrate solution was preparing by weighing 20.0 mg of FDA in 10.0 mL of analytical grade Acetone using a sterile 15 mL centrifuge tube. This solution was divided into 1 mL aliquots labeled as 4.8 mM FDA stock solution in sterile test tubes. The tubes were protected from light and stored at −20° C.

Substrate 4-Methylumbelliferyl-acetate Preparation

A weighed amount of 9.8 mg 4-Methylumbelliferyl-Acetate substrate was transferred to a sterile 15 mL centrifuge tube and completely dissolved in 10.0 mL ethanol. The solution was mixed, capped and labeled 44.9 mM 4-Methylumbelliferyl-Acetate stock solution. The solution was protected from light and stored at 4° C. Prior to use, a volume of 0.03 mL of this 44.9 mM stock was added to 9.97 mL ethanol to yield a final working concentration of 0.135 microMolar (uM).

HEPES Buffer Reagent Preparation

HEPES, N-[2-hydroxyethyl]piperazine-N′-[2-ethanesulfonic acid] Buffer (pKa=7.48), buffer was prepared and the pH was adjusted to 7.5 or 8.0. A volume of 0.25 mL of stock 1M HEPES was added to 45.0 mL nanopure grade autoclaved water and adjusted to pH=7.5 or 8.0 using 1M NaOH. The solution was diluted to 50 mL using nanopure grade sterilized water, protected from light, and kept at room temperature. The working HEPES buffer solution was prepared fresh daily.

### EXAMPLE 2

Bacteria

Bacteria used were obtained from American Culture Collection: E.coli 609 (ATCC 700609), Klebsiella pneumoniae (ATCC 27736), Salmonella typhimurium (ATCC 7007390), Alcalegenes faecalis (ATCC 8750), Mycobacterium phlei (ATCC 11758), Novosphingobium capsulatum (ATCC 14666), and Pseudomonas aeruginosa (ATCC 10145).

For growing E. coli, Klebsiella and Salmonella, trypticase soy broth (TSB) was used. For Alacaligenes, Novosphigobium and Pseudomonas, nutrient broth was used. For Mycobacterium, middlebrook broth supplemented with middlebrook ADC enrichment broth was used. A volume of 1.0 mL of each strain was taken from pure stocks and suspended in 9.0 mL of the corresponding broth media. The bacterial suspension was incubated in a shaker-incubator (New Brunswick Scientific C24, Edison, N.J.) (150 RPM at 37° C.) to achieve a log phase bacterial culture. Bacterial cultures were centrifuged for 10 minutes at 1000×g and 22° C. After centrifugation, supernatants were discarded. The remaining bacterial pellets were washed twice with phosphate buffer (0.5×PBS) and resuspended in 2.0 mL of the same buffer. Optical densities were measured for aliquots of 10⁻¹ to 10⁻³ serial dilutions using a spectrophotometer (Hach DR/4000U, Loveland, CO) at 600 nm.

In a separate study, mixed cultures of E coli (ATCC 700609) and Pseudomonas aeruginosa (ATCC 10145) were used. A volume of 1.0 mL of each strain was taken from pure frozen stocks and suspended in 9.0 mL of trypticase soy broth (TSB) media. The bacterial suspension was incubated in a shaker-incubator (150 rpm and 37° C.) to achieve a log phase bacterial culture. Log-phase bacterial cultures were centrifuged for 10 minutes at 1000×g force and 22° C. and supernatants were discarded. The remaining bacterial pellets were washed twice with phosphate buffer saline (0.5×PBS) and resuspended in 2.0 mL of the same buffer. Optical densities were measured for aliquots of 10⁻¹ to 10⁻³ serial dilutions using a spectrophotometer at 600 nm. Aliquots of the bacterial dilutions that yielded 0.2 optical densities were used for testing in this study. All bacterial dilutions, buffers, and substrates were made in autoclaved nanopure grade water.

Biofilm Reactors

For pure culture biofilm growth, biofilm reactors made of plastic housings supported by inlet and outlet ports were used. The reactors were soaked in 70% propanol overnight, dried, and rinsed three times with autoclaved nanopure grade water before experimental set up. A volume of 1.0 mL from each pure bacterial culture at an OD₆₀₀ 0.2 was pipetted into the corresponding batch reactor containing 325 mL of autoclaved tap water for glass coupon colonization and biofilm formation. Water was recirculated using peristaltic pumps (Cole-Palmer EW-78225, Vernon Hills, Ill.) set at 6 rpm. Glass coupons (VWR, West Chester, Pa.) were mounted on nylon combs and placed in the biofilm reactors and incubated for known periods of time. All biofilm reactors were sealed and kept at room temperature.

Coupon Preparation for Biofilm Formation

Glass coupons were cut and sized to fit the inner dimensions of a cuvette (1.25 cm×3.75 cm±0.1 cm). These coupons were soaked in 70% propanol solution overnight, dried, placed in nanopure grade water, and autoclaved before use. All coupons were mounted vertically on sterilized nylon combs and placed in sterile isolated biofilm reactors containing water inoculated with pure culture bacteria as described above. After a known incubation period, coupons colonized with pure culture biofilms were removed aseptically and analyzed using fluorescence.

Lipophylic, Metabolic, Proteolytic, and Glycosidic Assays

All assays were performed in total volume of 3.5 mL in the reaction cell. In most assays, the 3.5 mL volume consisted of 3.35 mL of HEPES buffer adjusted to pH 8.0±0.02, bacterial suspension (0.10 mL) at OD₆₀₀ 0.2, and 0.05 mL of the corresponding substrate. The assays were performed at room temperature (22-26° C.). Assay times were 5 min, 10 min, 30 min, and 120 min, for lipophylic, metabolic, proteolytic, and glycosidic activities, respectively. An Ocean Optics Spectrometer HR2000 was used to measure fluorescence emissions. The excitation wavelength was fixed for all assays in the range of 440-465 nm. Collection of signals were obtained at appropriate wavelength emissions.

For the mixed E. coli-Pseudomonas study, an equal volume (0.10 mL) of each strain was used in the reaction cell. The HEPES buffer volume was reduced to 3.25 mL in order to maintain a total volume of 3.5 mL in the reaction cell.

### EXAMPLE 3

Single Excitation-dual Emission Ratio Techniques (SEDERT) to Characterize Biofilms of E. coli and Mycobacterium phlei

E. coli and Mycobacterium phlei biofilms were grown as described above. The colonized coupon was then added to a reaction cell and assayed for proteolytic, glycosidic and lipophylic activity using the LLβN, MUF-β and NPN, respectively, reagents described above.

Each bacterium has a signature which is identified at specific emission wavelengths of 410, 440, and 420 nm for the proteolytic, glycosidic, and lipophylic assays, respectively. For example, E. coli has signatures of 0.56, 0.17 and 0.27 at 420, 440 and 410 nm. These “signatures” correspond to the linear slope of fluorescence emissions for each of the substrates tested. Similarly, the opportunistic pathogen Mycobacterium phlei signatures are 0.56, 0.21 and 0.23 at 420, 440 and 410 nm at 7 weeks of biofilms formation.

### EXAMPLE 4

Pure Culture Biofilms

A portable biosensing device comprising a light source, flexible fiber optic probe, optical lenses and filters, reaction cell and a spectrophotometer able to accept the fiberoptic probe to measure fluorescent light intensity was used to detect biofilms. The emitted light, fluorescence from the biofilm, was collected at the tip of the fiberoptic probe and was transferred to a spectrophotometer via a fiberoptic cable. The total fluorescent light intensity was evaluated from the emission spectrum by numerical integration.

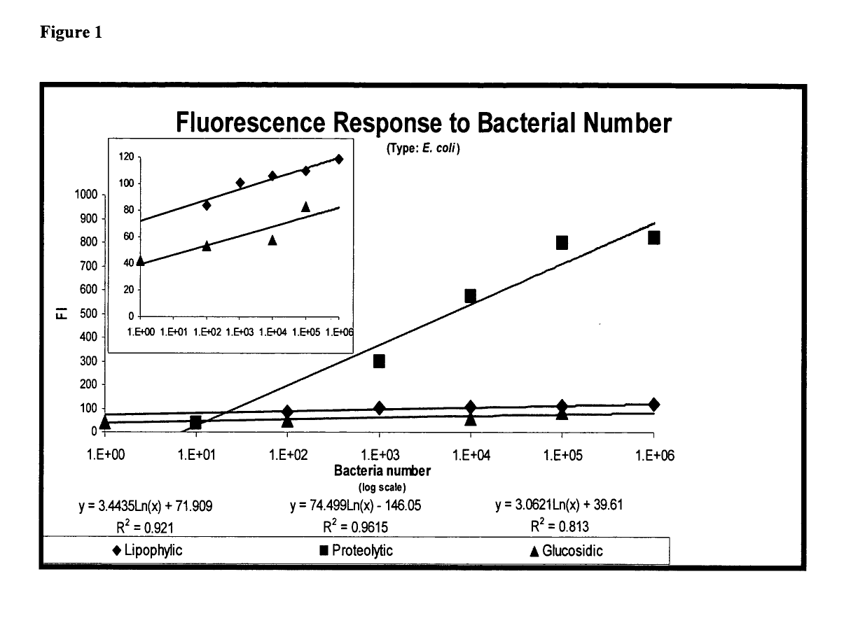

For signal optimization, pure cultures of planktonic E. coli were grown as described above and then assayed for proteolytic, glycosidic and lipophylic activity using the reagents described above (FIG. 1).

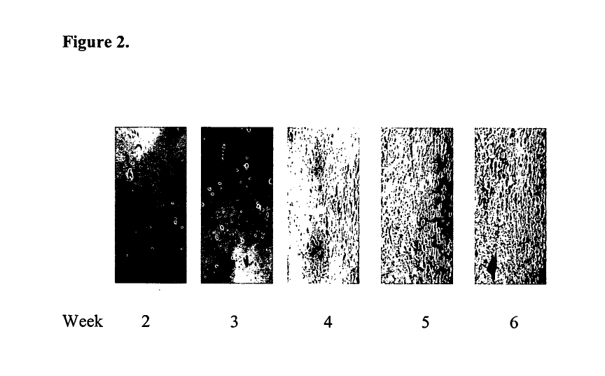

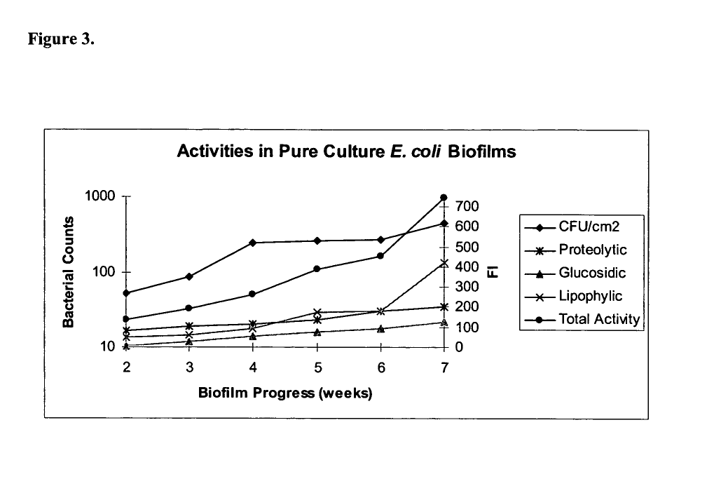

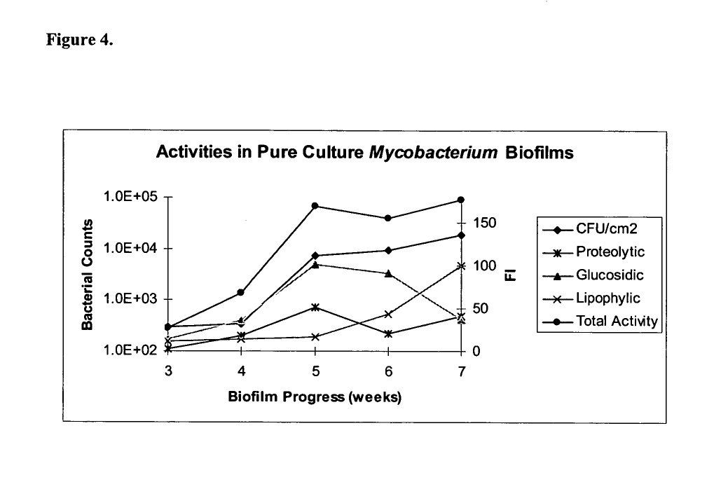

To study different stages of biofilm development, E. coli and Mycobacterium biofilms were generated as described above. Microscopic images of E. coli biofilms at different stages of development are presented in FIG. 2. A battery of assays was used to generate signature signals for biofilm detection based on specific biochemical assays to quantify biomolecules using fluorescence techniques. N-phenyl-2-naphthylamine, L-Leucine β-Naphthylamide and 4-Methylumbelliferyl β-D-glucopyranoside were used to quantify lipid, protein, and polysaccharide activities of microbial cells in biofilms, respectively (FIGS. 3 and 4).

In the optimization study, generated signals from lipophylic, proteolytic, and glycosidic assays showed a positive linear correlation with the number of microorganisms (E. coli) as evidenced by correlation coefficients of 0.92, 0.96, and 0.81, respectively (FIG. 1).

In the assays using E. coli and Mycobacterium biofilms, a linear increase in proteolytic and glycosidic activities was observed, whereas lipophylic activities showed an exponential increase over a period of several weeks (FIGS. 3 and 4).

### EXAMPLE 5

Single Excitation Single Emission Approach

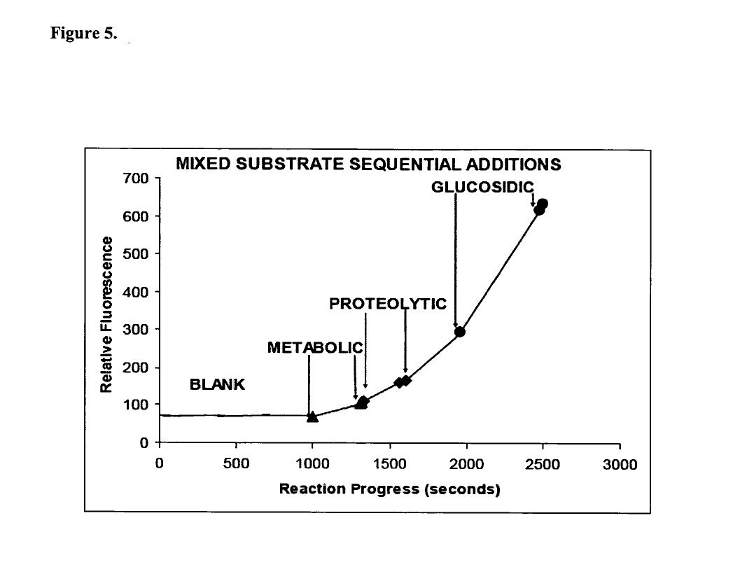

Novosphigobium cells were grown as described above and then assayed as described above for metabolic, proteolytic, and glycosidic activity using the reagents where all of the enzyme substrates were modified to have the same fluorescence-emitting molecule, methylumbelliferone (MUF). In particular, 4-Methylumbelliferyl-acetate, L-Leucine-4-Methylumbelliferone, and 4-Methylumbelliferyl-β-D-Glucopyranoside for metabolic, proteolytic, and glycosidic activities, respectively. Because all of the substrates have the same fluorescence-emitting molecule, the substrates must be added sequentially to detect the individual enzyme-substrate reactions. The use of the same fluorescence-emitting molecule is expected, however, to minimize variations due to the use of different fluorescence molecules and multiple excitation-emission wavelengths. The results show that multiple signals of multiple enzymatic activities can be collected and analyzed (FIG. 5).

### EXAMPLE 6

Pure cultures of planktonic bacteria were grown as described above and then assayed as described above for their enzymatic activities such as metabolic, proteolytic, glycosidic using the FDA, LLβN and MUF-β reagents described above. The fluorescence assays were performed to identify characteristic enzymatic activities in suspended bacteria. Each bacterial group expressed unique enzymatic reaction patterns as shown in Table 1a (reported as Relative Fluorescence Intensity (“RFI”)) and specific biochemical reaction signatures Table 1b ((RFI (metabolic)+(RFI (proteolytic)+RFI (glycosidic)/RFI (metabolic or proteolytic or glycosidic)).

TABLE 1a
Pattern of Enzymatic Activities in Different Bacteria.

| Bacteria | Metabolic | Proteolytic | Glycosidic |
|---|---|---|---|
| E. coli | 202 | 23 | 15 |
| Alcaligenes | 217 | 4 | 0 |
| Novosphingobium | 256 | 163 | 225 |
| Pseudomonas | 206 | 27 | 299 |
| Salmonella | 220 | 9 | 0 |

TABLE 1b
Planktonic Characterization-Signature

| Bacteria | Metabolic | Proteolytic | Glycosidic |
|---|---|---|---|
| E. coli | 0.84 | 0.10 | 0.06 |
| Alcaligenes | 0.98 | 0.02 | 0.0 |
| Novosphingobium | 0.40 | 0.25 | 0.35 |
| Pseudomonas | 0.39 | 0.05 | 0.56 |
| Salmonella | 0.96 | 0.04 | 0.0 |

### EXAMPLE 7

Validation of the Assay through Identification of Unknown Samples

The fluorescence assays were performed on unknown water samples to identify bacterial species using enzymatic activities. The cultures were grown as described above and then assayed as described above for their enzymatic activities such as metabolic, proteolytic, glycosidic using the FDA, LLβN and MUF-β reagents described above. Each bacterial group expressed unique enzymatic reactions shown in Table 2 (reported as Relative Fluorescence Intensity (“RFI”)).

TABLE 2
Identification of Unknown Bacteria Using Specific Signature.

| | | Proteo- | | Conformity |
|---|---|---|---|---|
| Bacteria | Metabolic | lytic | Identification | level |
| E. coli | 60 | 44 | | |
| Novosphingobium | 1000 | 326 | | |
| Salmonella | 57 | 1 | | |
| Pseudomonas | 108 | 16 | | |
| Alcaligenes | 63 | 5 | | |
| Unknown0 | 1000 | 326 | Novosphingobium | 100% |
| Unknown1 | 61 | ND | Salmonella | 100% |
| Unknown2 | 62 | 6 | Alcaligenes | 100% |
| Unknown3 | 120 | 27 | Pseudomonas | 100% |
| Unknown4 | 63 | 51 | E. coli | 100% |

The above data clearly demonstrate that different bacteria can be quickly characterized and identified using the methods of the invention.

It should be understood that the foregoing disclosure emphasizes certain specific embodiments of the invention and that all modifications or alternatives equivalent thereto are within the spirit and scope of the invention as set forth in the appended claims.

1. A method for detecting bacteria in a sample comprising:
a. contacting a test sample with one or more fluorescence-emitting substrate selected from the group consisting of metabolic, proteolytic, glycosidic, and lipophylic substrates, under conditions suitable for the one or more substrates to be acted upon by bacterial enzymes present in the test sample that are specific for the one or more substrates;
b. detecting an activity over time of fluorescence emissions from one or more of the substrates that result from the contacting with the test sample; and
c. correlating the activities with the presence of live bacteria in the test sample.

2. The method of claim 1, wherein the contacting comprises contacting the test sample with two or more of the fluorescence-emitting substrates.

3. The method of claim 1, wherein the contacting comprises contacting the test sample with three or more fluorescence-emitting substrates.

4. The method of claim 1, further comprising:
d. contacting test samples identified as having live bacteria with one or more different fluorescence-emitting substrates selected from the group consisting of metabolic, proteolytic, glycosidic, and lipophylic substrates, under conditions suitable for the one or more different substrates to be acted upon by bacterial enzymes present in the test sample that are specific for the one or more different substrates;
e. detecting an activity over time of fluorescence emissions from one or more of the different substrates that result from the contacting with the test sample; and
f. correlating the activities with a bacterial type present in the test sample.

5. The method of claim 4, wherein the contacting comprises contacting the test sample with two or more of the different fluorescence-emitting substrates.

6. The method of claim 4, wherein the contacting comprises contacting the test sample with three or more different fluorescence-emitting substrates.

7. A method for detecting bacteria in a sample comprising:
a) contacting a test sample with one or more fluorescence-emitting substrates selected from the group consisting of metabolic, proteolytic, glycosidic, and lipophylic substrates, under conditions suitable for the one or more substrates to be acted upon by bacterial enzymes present in the test sample that are specific for the one or more substrates;
b) detecting an activity over time of fluorescence emissions from one or more of the substrates that result from the contacting with the test sample; and
c) correlating the activities with a bacterial type present in the test sample.

8. The method of claim 7, wherein the contacting comprises contacting the test sample with two or more of the different fluorescence-emitting substrates.

9. The method of claim 7, wherein the contacting comprises contacting the test sample with three or more different fluorescence-emitting substrates.

10. The method of claim 1, wherein the fluorescence-emitting substrates emit fluorescence at spectrally distinguishable wavelengths.

11. The method of claim 1, wherein the fluorescence-emitting substrates emit fluorescence at spectrally non-distinguishable wavelengths.

12. The method of claim 1, wherein detecting the activity over time of fluorescence emissions comprises sequentially detecting an activity over time of fluorescence emissions of fluorescence emission from each substrate.

13. The method of claim 1, wherein detecting the activity over time of fluorescence emissions comprises simultaneously detecting an activity over time of fluorescence emissions of fluorescence emission from each substrate.

14. The method of claim 1, wherein detecting the activity over time of fluorescence emissions comprises detecting a slope of fluorescence emissions.

15. The method of claim 1, wherein the test sample comprises a liquid sample.

16. The method of claim 12, wherein the liquid sample is derived from a sample selected from the group consisting of water samples, body fluid samples, beverage samples, drug samples, food samples, environmental samples, solid waste samples, and diagnostic samples.

17. The method of claim 1, wherein the test sample comprises a biofilm sample.

18. The method of claim 1, wherein the test sample comprises a gas sample.

19. The method of claim 1, wherein the contacting occurs at a controlled temperature.

20. The method of claim 1, wherein the contacting occurs at in a controlled light environment.

21. The method of claim 1, wherein the method is automated.

22. The method of claim 18, further comprising storing data obtained in a database.

23. A reaction cell comprising one or more fluorescence-emitting substrates selected from the group consisting of metabolic, proteolytic, glycosidic, and lipophylic substrates.

24. The reaction cell of claim 20, comprising two or more fluorescence-emitting substrates.

25. The reaction cell of claim 21, wherein the two or more fluorescence-emitting substrates are provided as powders, liquids, capsules, liquid granules, coatings on the reaction cell or any combination thereof.

26. The reaction cell of claim 21, further comprising a means for identification of the reaction cell.

27. A detection device, comprising: a light source for illuminating a sample; an optical filter; a sample compartment comprising a reaction cell and a fluorescence intensifier; and a fluorescence collection device adapted for connection to a fluorescence detector, wherein the optical filter is interposed between the light source and the sample compartment, and wherein the fluorescence intensifier is interposed between the reaction cell and the fluorescence collection device.

28. A computer readable storage media for automatically carrying out the methods of claim 1.

29. A fluorescence detection device comprising the computer readable storage media of claim 25.
